(19) United States
(12) Patent Application Publication
(10) Pub. No.: US 2024/0190648 A1
(43) Pub. Date: Jun. 13, 2024

(54) Surface Mount Air Disinfectant/Deodorizer Holder-Ejector Device.

(71) Applicant: STEPHEN NWADIOGBU OKUDO, Suwanee, GA (US)

(72) Inventor: STEPHEN NWADIOGBU OKUDO, Suwanee, GA (US)

(21) Appl. No.: 18/076,892

(22) Filed: Dec. 7, 2022

(51) Int. Cl. B65F 1/14

(52) U.S. Cl. CPC B65F 1/14; B65F 2210/129

(57) ABSTRACT

Devices, systems, and methods are disclosed for facilitation of continuous release of fluids or vaporizable solids using disinfectants, deodorizers, repellants, etc., into an enclosed unit, such as trash-can in consistent and predictable amounts. The device generally includes a surface mount holder-ejector device in the resemblance of letter “H”; inward slanted upper arm as in the upper arm of letter “H”; inward slanted lower arm as in lower arm of letter “H”; or slightly curve bilateral arms as in letter “H” that may have thumb-finger fitting. The device has an encasement unit with holder-ejection mechanism mounted on the horizontal arm of the device as in the horizontal arm of letter “H”. Device can be utilized with modified disposable or refillable fluid container cartridge adaptable to the device, and device surface wall affixer. The lower “H” shaped arms of the device may be thumb-finger compressed to cause outward pull of the two upper “H” shaped arms of the device to allow insertion or ejection process of the cartridge. The ends of the two upper arms of the device have inward projection forming stoppers/internal grip surface that causes retention of the cartridge in the device and restricting outward pressure by the spring and flat-plate beneath the cartridge.

### CROSS-REFERENCE TO RELATED APPLICATIONS

Not Applicable

### BACKGROUND OF THE INVENTION

### Field of the Invention

The present disclosure relates to surface mount air freshener/disinfectant/deodorizer holder-ejector device in particular; to maintain retention of the device, retention of the device adapted air-conditioning product container, and overall device operation of air freshening/disinfecting/deodorizing while mounted onto a movable lead of an enclosable unit.

### Background Art

Trash-can sanitization has been a continuing challenge for essentially as long as there had been trash-can containers. Over the years, many different techniques and devices have been developed, particularly for trash-can sanitization and odor control. Some of the challenges include maintaining continuous dissemination of disinfectant/deodorizer and retainment of device during removal-replacement of trash-bag or emptying of trash-can. The present invention is typically made of plastic material, remains reusable, allows for product replacement with minimal user exposure to contamination.

Despite the many different techniques and devices that have been developed, there is a continuing long felt need in the industry for improved trashcan disinfecting/deodorizing device and methods.

### BRIEF SUMMARY OF THE INVENTION

Accordingly, the present invention provides devices and method for disinfecting/deodorizing an enclosed unit, such as disinfecting/deodorizing trash-can container using; one or more of disinfectants, deodorizers, insect repellants, etc., in an enclosed unit, such as trash-can, in consistent and controlled amounts. Devices generally include a holding and ejecting mechanism to keep in place dispensation of disinfectants, deodorizers, insect repellants, etc. into the enclosed unit and for the release of product container after product finish while remain in place for device reuse.

In various embodiments, the device may include a disposable or refillable cartridge and device surface wall affixer. 1) Device may have the resemblance of letter “H”; inward slanted upper arm of letter “H”; inward slanted lower arm of letter “H”; or slightly curve bilateral arms of letter “H”, such that any of these configurations may be thumb-finger fitting. 2) The lower “H” shaped arms of the device may be thumb-finger compressible to cause outward pull of the two upper “H” shaped arms of the device. 3) The ends of the two upper arms of the device have inward projection forming stoppers or grip internal surface that are opposite in direction at rest position. 4) A top-flanged cup fitted on the horizontal beam connecting the two vertical arms of the device. 5) Out-stretched spring fitted to the interior center base of the top-flanged cup that suspends a flat-plate which may freely move in the cup but restricted by the cup flange. 6) Inverted letter “L” shaped cup holder attached to flanged cup and may be located on the cup-side perpendicular to the side of the two letter “H” shaped arms of the device. 7) A hollow cubic/cuboid surface affixable cup holder into which top-flanged cup holder is placed to secure device to the interior surface wall of the enclosed unit.

### BRIEF DESCRIPTION OF THE DRAWINGS

The accompanying drawings are included for the purpose of exemplary illustration of various aspects of the present invention to aid in description, and not for purposes of limiting the invention, wherein:

FIG. 1-6. illustrates a cross-sectional front view of exemplary embodiments of the device of various configuration in neutral positions. Where FIG. 1. Illustrates a cross sectional front view of the device (20); Surface affixable cup holder (30); and accessorial device cartridge (10).

FIG. 2. illustrates a cross-sectional front view of exemplary embodiment of the device in a neutral position where the ends of the two upper arms of the device have inward projection forming upward slanted stoppers or grip internal surface that are opposite in direction.

FIG. 3. illustrates a cross-sectional front view of exemplary embodiment of the device in a neutral position where the ends of the two upper arms of the device have inward projection forming upward slanted and ball-ended stoppers or grip internal surface that are opposite in direction.

FIG. 4. illustrates a cross-sectional front view of exemplary embodiment of the device in a neutral position where the ends of the two upper arms of the device have inward projection forming upward slanted and ball-ended stoppers or grip internal surface that fit into the socket feature of configured cartridge container.

FIG. 5. illustrates a cross-sectional front view of exemplary embodiment of the inward slanted upper arm device configuration in a neutral position where the ends of the two upper arms of the device have inward projection forming flat stoppers or grip internal surface that are opposite in direction.

FIG. 6. illustrates a cross-sectional front view of exemplary embodiment of the inward slanted upper arm device configuration in a neutral position where the ends of the two upper arms of the device have inward projection that forms upward slanted stoppers or grip internal surface that are opposite in direction.

FIG. 7. illustrates a cross-sectional front view of exemplary embodiment of the inward slanted lower arm device configuration in a neutral position where the ends of the two upper arms of the device have inward projection forming flat stoppers or grip internal surface that are opposite in direction.

FIG. 8. illustrates a cross-sectional front view of exemplary embodiment of the inward slanted lower arm device configuration in a neutral position where the ends of the two upper arms of the device have inward projection forming slanted stoppers or grip internal surface that are opposite in direction.

FIG. 9. illustrates a cross-sectional front view of exemplary embodiments of the device in a neutral position with numeral identification.

FIG. 10. illustrates a three-dimensional sectional view of exemplary embodiments of the device in a neutral position with numeral identification and description of component parts. As shown in FIG. 10, device (20) component description is as follows: (20A) is the device upper arms with stopper/grip projections (21); (20B) is the device lower arms with end part (25); (22) is the space between the stoppers; (23) is the device top-flanged cup; and (24) is the device horizontal arms connecting vertical arms to give the appearance of letter “H”.

FIG. 11. illustrates across-sectional side view of exemplary component of the device top-flanged cup (23) with top-flanged cup holder (23-ii); out-stretched spring (23-iv) fitted to the interior center base (23-v) of the bottom part (23-vi) of the top-flanged cup; further, top part of the out-stretched spring suspends a flat-plate (23-iii) that may freely move in the cup but restricted by the cup flange (23-i).

FIG. 12 illustrates a cross-sectional side view of exemplary device top-flanged cup (23) with Out-stretched spring (23-iv) compressed due to pressing down of the flat-plate (23-iii).

FIGS. 13 & 14. illustrated an exemplary of the device in three-dimensional appearance of top-flanged cup which is a component part of the device.

FIGS. 15, 16, & 17. Illustrated cross sectional view of exemplary embodiment of accessorial modified dispenser cartridge comprises of cylindrical, cubic, or cuboid container, etc (11) embedded into solid thick flat-brim (14) semi-oval shaped cup (15). A fluid filled cartridge may have an inbuilt perforated (13) funnel-packed absorbent or a stick of absorbent (12) that may extending out of the container to facilitate fluid to evaporate out of the fluid container.

FIG. 15. Illustrated cross sectional view of exemplary embodiment of accessorial modified dispenser cartridge comprises of cylindrical, cubic, or cuboid container where the holder is in a socket configuration.

FIG. 16. Illustrated cross sectional view of exemplary embodiment of accessorial modified dispenser cartridge comprises of cylindrical, cubic, or cuboid container where the holder is in a flat brim-top configuration.

FIG. 17. Illustrated cross sectional view of exemplary embodiment of accessorial modified dispenser cartridge comprises of cylindrical, cubic, or cuboid container where the holder is in a flat brim-top configuration but consist of a funnel-packed absorbent or a stick of absorbent (12) that may extending out of the container to facilitate fluid to evaporate out of the fluid container.

FIGS. 18, 19, & 20. Illustrates exemplary embodiment of device component part, the surface affixable cup holder, into which top-flanged cup holder is placed to secure device to the interior surface wall of the enclosed unit. In further description, FIG. 18. illustrates a cross-sectional top view of the surface affixable cup holder; FIG. 19. illustrates a lateral side view of the surface affixable cup holder; and FIG. 20. Illustrates a three-dimensional view of the surface affixable cup holder.

FIG. 21. illustrates a cross-sectional front view of exemplary device thumb-finger compression by the lower “H” shaped arm (20B)/(25) of the device causing the upper “H” shaped arm (20A) to pull back for insertion of the accessorial oval-base cartridge (10).

FIG. 22. illustrates cross-sectional front view of exemplary device thumb-finger release of the lower “H” shaped arm (20B)/(25) of the device causing the upper “H” shaped arm (20A) to return to rest securing accessorial cartridge by device stoppers (21) resting against cartridge flat-brim/grippable surface (14). Further, as shown in FIG. 10, cartridge insertion causes suspended flat-plate depression and out-stretched spring compression.

FIG. 23. illustrates a cross-sectional front view of exemplary device thumb-finger compression of lower “H” shaped-arm (20B/25) causing upper “H” shaped arm (20A) to pull back, device stopper (21) disengagement from cartridge flat-brim top (14).

FIG. 24. illustrates a cross-sectional front view of exemplary embodiments of the device thumb-finger compression by the lower “H” shaped arm (20B)/(25) of the device causing re-expansion of out-stretched spring (23-iv), flat-plate (23-iii) rising, and upward thrust of cartridge.

FIG. 25. illustrates a cross-sectional frontal view of exemplary embodiments of the device at complete rest state; re-expansion of out-stretched spring; and flat-plate suspension at elevated level restricted by cup flange.

FIG. 26. Illustrates the exemplary embodiments of a cross-sectional front view (10/20) of the device (20), and FIG. 27, a cross-sectional side view (10/20/23-ii/31) of same device (20) with device components: top-flanged cup holder (23-ii) and surface affixable cup holder (30), and accessorial air-freshener cartridge (10). It will be appreciated if surface affixable cup holder is mounted to the desired surface prior to insertion of top-flanged cup holder into surface affixer.

FIG. 27. illustrates a lateral-sectional view of exemplary embodiments of the device (20); depicting top-flanged cup holder (23-ii) and surface affixable cup holder (30), and accessorial air-freshener cartridge (10). Of note, FIG. 27, presents device (20) and (10) in a pre-assembly stage.

FIG. 28. illustrates a cross-sectional front view, and cross-sectional side view, FIG. 29, of exemplary embodiments of the device top-flanged cup holder inserted into surface affixable cup holder.

FIG. 29. illustrates a lateral-sectional side view of exemplary embodiments of the device top-flanged cup holder inserted into surface affixable cup holder with its accessory (10). Of note, FIG. 29, presents device (20) and (10) in a post-assembly stage.

FIG. 30. illustrates a cross-sectional side view of exemplary embodiments of the surface mount air-freshener holder-ejector device (20) mounted upside-down position into surface affixable cup holder (30) that is installed via adhesive compound onto the lead cover (40-i) of an enclosable unit (40) such as trash-can.

In the drawings and detailed description, the same or similar reference numbers may identify the same or similar elements. It will be appreciated that the implementations, features, etc. described with respect to embodiments in specific figures may be implemented with respect to other embodiments in other figures, unless expressly stated, or otherwise not possible.

### DETAILED DESCRIPTION OF THE INVENTION

Surface mount air disinfectant/deodorizer holder-ejector device (20) with modified fluid container-dispenser adaptable to surface mount air disinfectant/deodorizer holder-ejector device (10), and affixable holder (30).

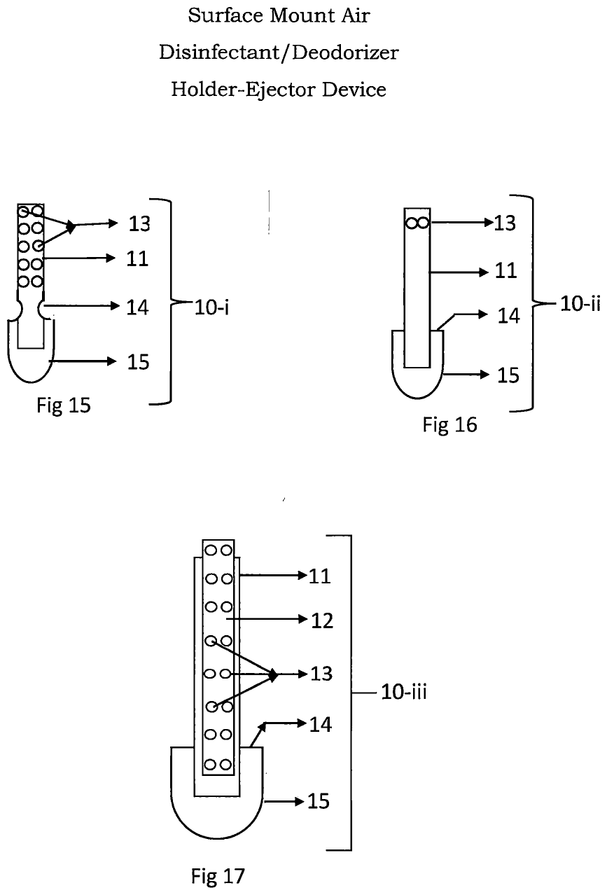

In various embodiment, device accessory such as cartridge (10) may be disposable or refillable non-disposable unit and can be liquid filled or vaporizable solid fill. As shown in FIGS. 15 & 16, device accessory comprises of cylindrical, cubic, or cuboid container, etc (11) embedded into solid thick flat-brim or socket (14) oval shaped base cup (15). A fluid filled cartridge may have an inbuilt perforated (13) funnel-packed absorbent or a stick of absorbent (12) extending out of the container to facilitate fluid to evaporate out of the fluid container.

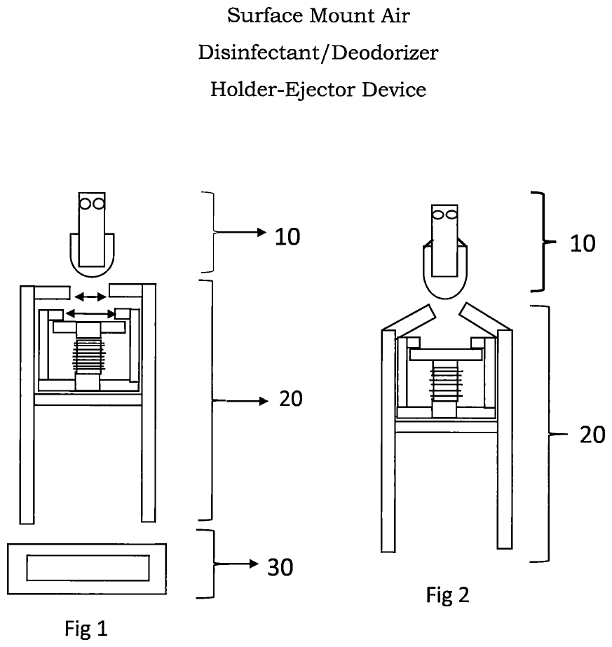

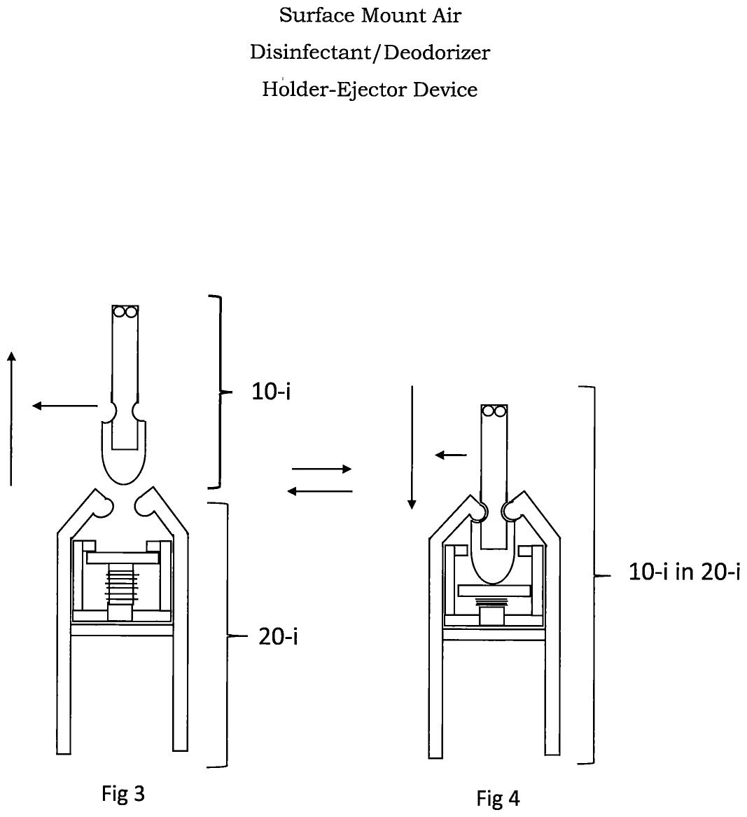

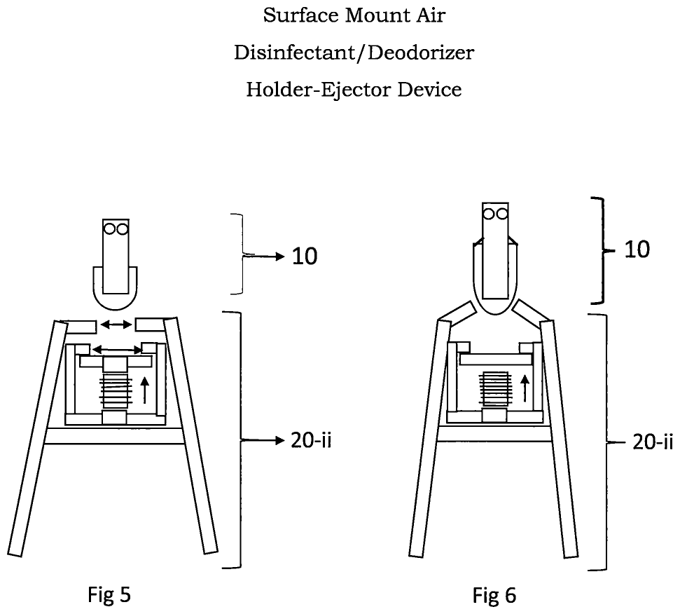

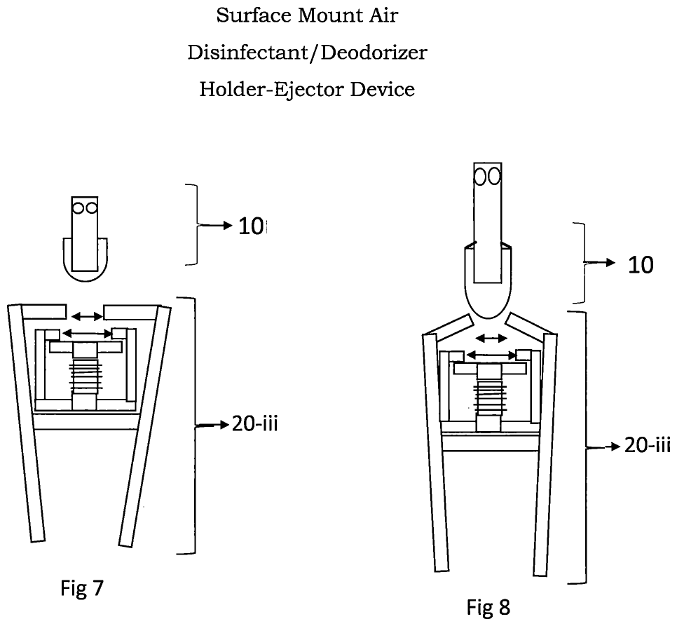

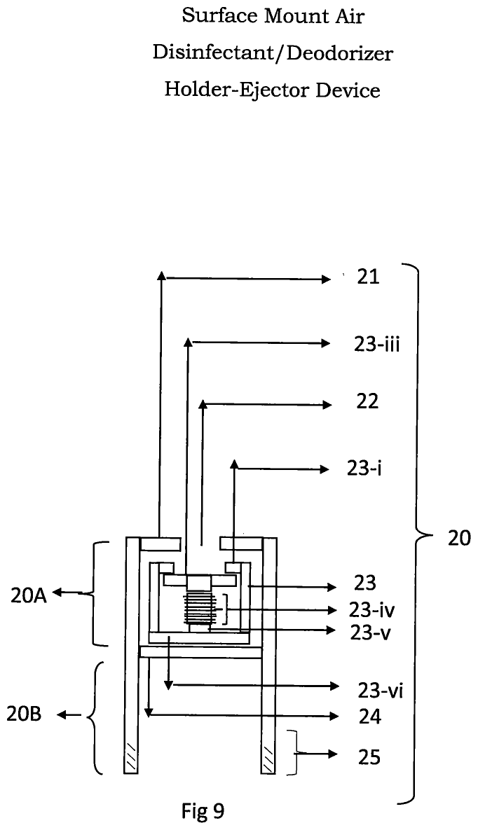

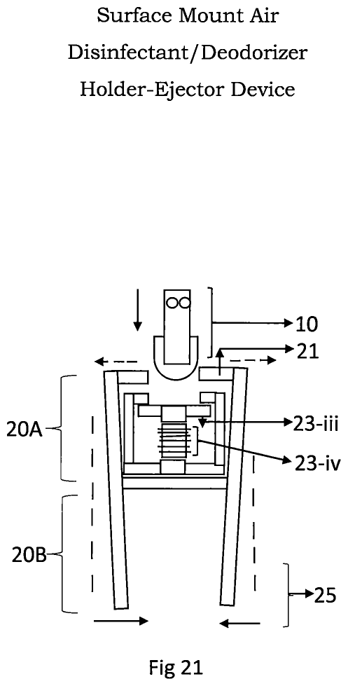

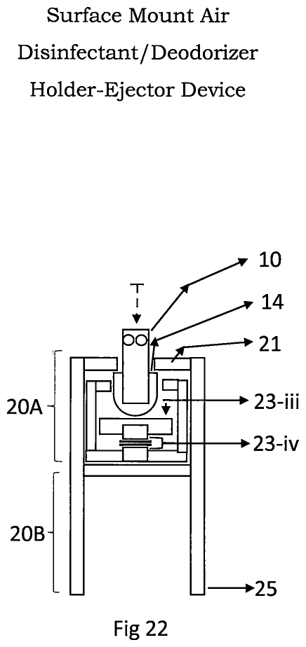

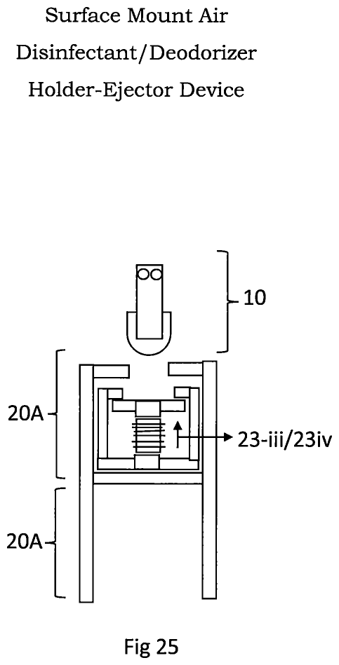

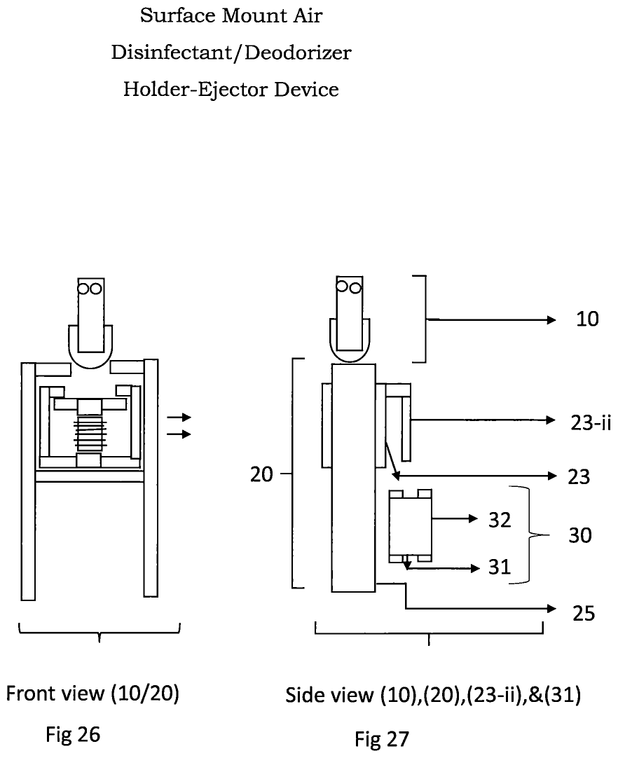

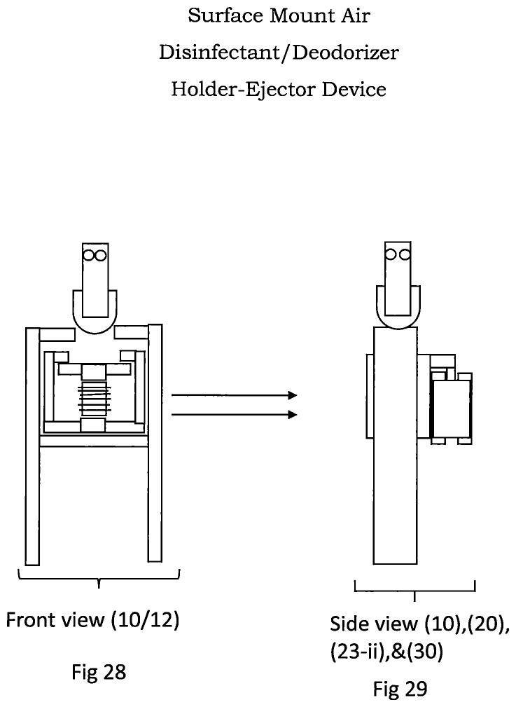

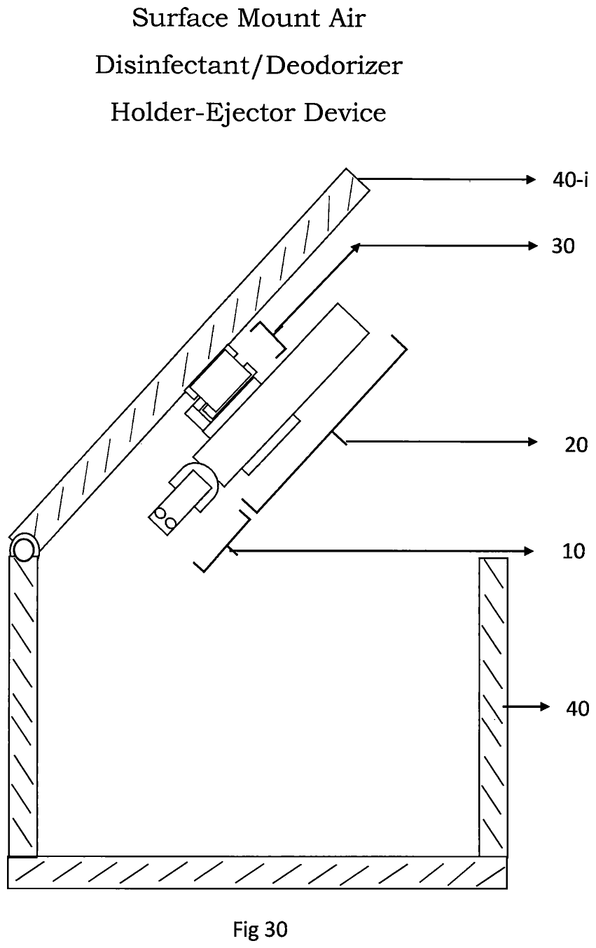

In this embodiments, air disinfectant/deodorizer holder-ejector device (20) may take the appearance of letter “H” (FIG. 1-4); inward slanted upper arm of letter “H” (FIGS. 5 & 6); inward slanted lower arm of letter “H” (FIGS. 7 & 8); or slightly curved bilateral arms of letter “H”. As shown in the afore mentioned illustrations, the upper “H” arms (20A) of the device may have inward projection forming stopper ends (21) or coarse grippable inner surface of the end of the upper “H” arm (20A) of the device. The lower “H” arm (20B) of the device may be thumb-finger compressible ends (25) that will result in the outward extension (pulling out) of the upper arm along with stopper ends (20A/21) away from the center (22) as shown in FIG. 21-25.

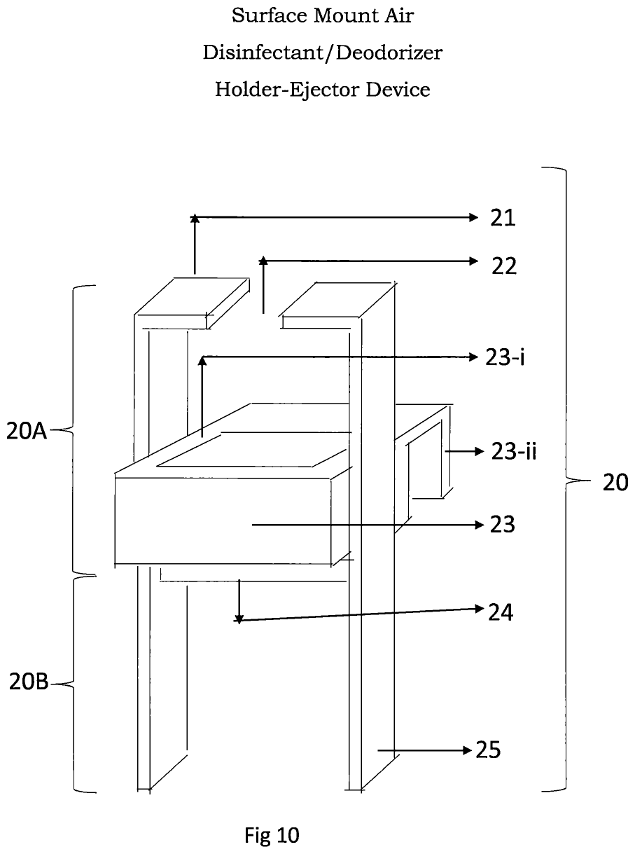

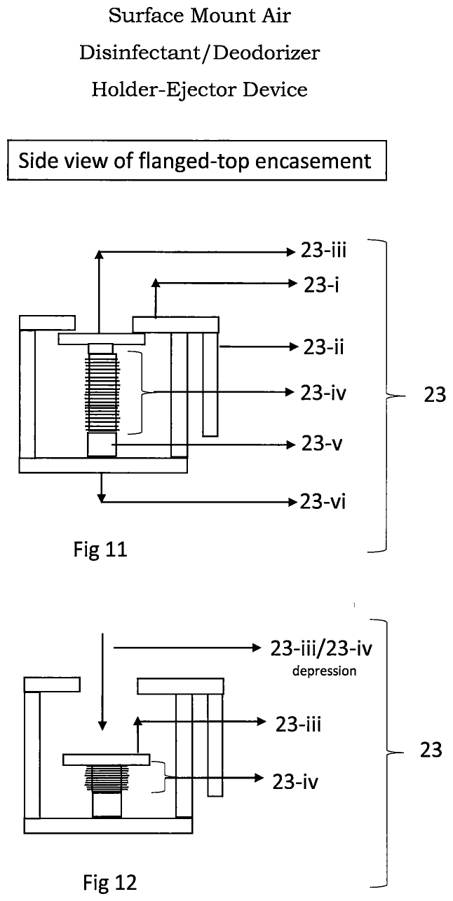

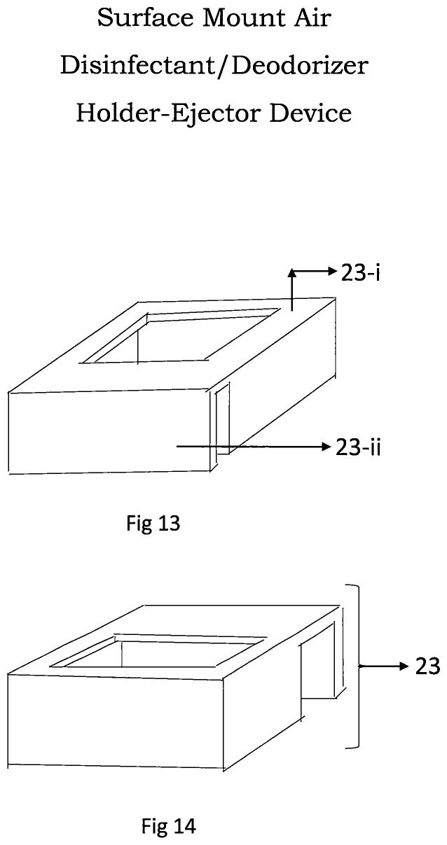

As illustrated in FIG. 1-8, a cross-sectional front view, and in FIG. 10, a three dimensional-sectional view of exemplary embodiments of the device, a flanged top cup (23) tightly fitted on horizontal beam (24) connecting both arm of the device as in vertical “H” shaped arms of the letter “H”. As shown in FIGS. 11 & 12, an inverted letter “L” shaped cup holder (23-ii) located on the cup-side surface that is perpendicular to side surface location of letter “H” shaped arms of the device. The top-flanged cup holder (23-ii) dimensions may be penetrable to hollo space (31) of the surface affixable cup holder (30), securely fit in surface affixer, and can be remove with ease.

In various embodiment, as shown in FIGS. 11 & 12, out-stretched spring (23-iv) secured to the base (23-v) of the top-flanged cup firmly suspends a flat-plate (23-iii). As illustrated in various figures (11,12,21-25), the flat-plate is of same shape as the internal shape of the top-flanged cup and rests against the cup flange (23-i) with pressure. As illustrated in Figures (11,12,21-25) of the cross-sectional side/front views of exemplary embodiments of the device, the movement of spring suspended flat-plate is free within the top-flanged cup except restriction by the flange.

The skilled artisan will appreciate that while the depth of the device top-flanged cup accommodates spring-plate compression and a proportional length of the cartridge, the cup flange does not restrict the cartridge insertion and the ejection movement processes.

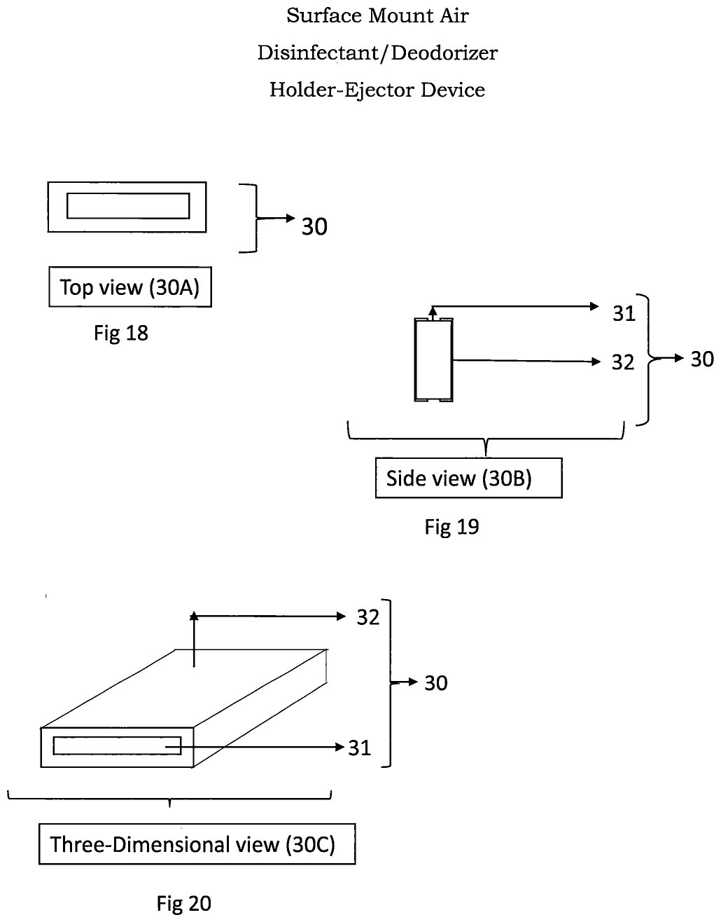

FIGS. 18,19, & 20. illustrates removable or non-removable surface affixable cup holder of the device (30) that has two surfaces (32) and a central hollo (31) that has same shape and dimensions as the cup holder (23-ii). In various embodiment, the surface affixable cup holder can be mounted via adhesive compound to any desired surface or on the internal surface of the enclosed unit. The top-flange cup holder (23-ii) of the device may easily slide into the hollo aspect (31) of the affixable cup holder and remained secure.

In various embodiments, the cartridge may be place into the device by thumb-finger compression of the lower “H” shaped arms (20B/25) causing the upper “H” shaped arm (20A) to pull outward along with the stopper (21) to allow placement of cartridge by semi-oval base (15) into device cup (23). Insertion force of cartridge by semi-oval base causes spring suspended flat-plate (23-iii) to compress the spring (23-iv) as illustrated in FIGS. 11 and 12. Upper “H” shaped arm of the device (20A) returns to rest along with both stoppers (21) resting against the flat-brim top/socket (14) of solid cup base of the cartridge, thereby countering spring-plate opposing outward pressure.

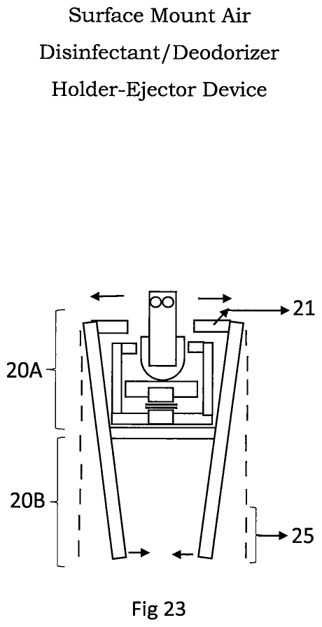

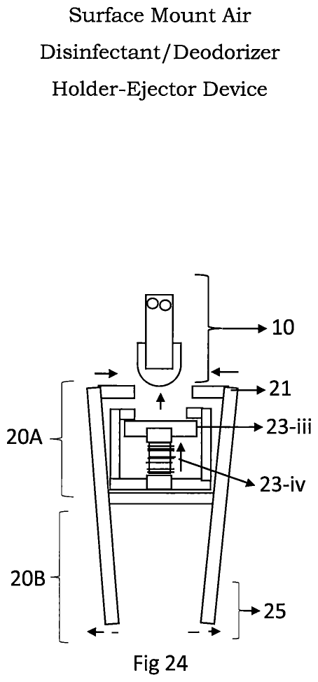

As illustrated in FIGS. 23 & 24, cross-sectional front view of exemplary embodiments of the device, cartridge ejection operation begins by thumb-finger compression of the lower “H” shaped arms (20B/25) of the device causing simultaneous outward pull of the upper “H” shaped arms (20A) and stoppers (21). Subsequently allowing the spring (23-iv) to stretch out pushing the flat-plate (23-iii) to ejecting cartridge out of the device cup (23). As illustrated in FIGS. 24 & 25, removal of thumb-finger compression causes upper (20A) and lower (20B) “H” shaped arms of the device to returns to rest. It is generally desirable to install the device upside-down on the internal cover of the trash-can, as an example, so that cartridge ejection will direct the cartridge into the trash-can.

It is to be understood that the above description is intended to be illustrative, and not restrictive. Many other embodiments will be apparent to those skilled in the art, upon reviewing the above description. The scope of the invention should, therefore, be determined with reference to any appended claims, along with the full scope of equivalents to which such claims are entitled.

Some implementations are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set. Also, as used herein, the terms “has,” “have,” “having,” or the like are intended to be open-ended terms.

The presence or absence of a summary, abstract, or claims in this application should in no way be considered as limiting on the scope of any inventions disclosed herein.

What is claimed is:

1. The device of claim, where the device takes appearance of the letter “H” shape that is thumb-finger grip fitted with encased holder-ejector unit mounted on the horizontal arm of the device as on the horizontal arm of letter “H”.

2. A device according to claim 1, wherein the device is in the resemblance of letter “H” shape; inward slanted upper arm of letter “H” shape; inward slanted lower arm of letter “H” shape; or slightly curve bilateral arms of letter “H” shape. Each configuration with encased holder-ejector unit mounted on the horizontal arm of the device as on the horizontal arm of letter “H”.

3. A device according to claim 1, wherein holder-ejector unit mounted on the horizontal arm of the device such that holder-ejector component of the device comprised of top-flanged cup, out-stretched spring fitted to the interior center base of the top-flanged cup, free movable flat-plate suspended to the opposite end of spring, and the flat-plate restricted by flange of the encasement.

4. A device according to claim 1, wherein the method of device operation is thumb-finger compressible two lower “H” shaped arms of the device such as to cause outward pull of the two upper “H” shaped arms of the device and the release of the two lower “H” shaped arms of the device will cause the two upper “H” shaped arms of the device to retract/return to original rest position.

5. A device according to claim 1, wherein the ends of the two upper “H” shaped arms of the device have inward projection forming stoppers or grippable inner surface ends that substantially causes retention of removable cartridge when inside the encased holder-ejector unit.

6. A device according to claim 3, wherein holder-ejector mechanism consists of:
(i) Out-stretched spring fitted in the interior base of the top-flanged cup of the holder-ejector unit mounted on the horizontal arm device
(ii) The opposite end of spring suspends a flat-plate that can freely move in the top-flanged cup of the device upon downward pressure but at rest is restricted by the encasement flange.

7. A device according to claim 3, wherein cartridge insertion into the encased holder-ejector unit mounted on the horizontal arm device is by either of mechanism listed below.
(i) Pressure application to the spring suspended flat plate using semi-oval end of the cartridge.
(ii) Thumb-finger compression of the lower “H” shaped arms of the device, causing outward pull of the upper arms of the device to allow insertion of the cartridge.
Of note, cartridge is retained into the holder-ejector unit by means of the stoppers/grip surface at the ends of the upper arms of the device after device arms are at rest on the cartridge.

8. A device according to claim 3, wherein cartridge ejection from the encased holder-ejector unit mounted on the horizontal arm device is by:
(i) Thumb-finger compression of the lower “H” shaped arms of the device, causing outward pull of the upper arms away from the center of the encased holder-ejector unit mounted on the horizontal arm device.
(ii) claim segment 8(i) resulting in spring expansion and corresponding upward movement of the flat plate to eject cartridge out of the encased holder-ejector unit mounted on the horizontal arm device.
(iii) The release of lower “H” shaped arms of the device, causing return of the upper “H” shaped arm to a rest.

9. A device according to claim 1, wherein the device facilitates continuous operation of air conditioning within enclosed environment and remains in place during trash-bag removal, replacement, or trash-can emptying process.

10. A device according to claim 1, wherein:
(i). One device mounted may be used for refillable and multiple non-disposable cartridge units.
(ii). Cartridge disposal performed by thumb-finger compression of the lower “H” arm of the device.
